(19) United States
(12) Patent Application Publication
(10) Pub. No.: US 2008/0268078 A1
(43) Pub. Date: Oct. 30, 2008

(54) Composition for inhibiting nitric oxide and/or prostaglandin E2 synthesis and method for inhibiting inflammation

(75) Inventors: Yu-Wen Su, Changhua County (TW); Meng-Hwan Lee, Yunlin County (TW)

Correspondence Address: BIRCH STEWART KOLASCH & BIRCH, PO BOX 747, FALLS CHURCH, VA, 22040-0747, US

(73) Assignee: Yangsen Biotechnology Co., Ltd.

(21) Appl. No.: 11/905,692

(22) Filed: Oct. 3, 2007

(30) Foreign Application Priority Data
Apr. 26, 2007 (TW) 096114758

(51) Int. Cl. A61K 36/00; A61K 31/045; A61K 36/23; A61K 36/738; A61P 29/00; A61K 36/899; A61K 36/232; A61K 31/365

(52) U.S. Cl. 424/765; 424/725; 514/470; 514/739

(57) ABSTRACT

A method for inhibiting nitric oxide and/or prostaglandin E₂ synthesis. The method comprises administering a composition to a subject, wherein the composition comprises an effective amount of butylidene phthalide, citronellol, geraniol or combinations thereof, which can be used to reduce or relieve the syndromes of the inflammation.

### BACKGROUND OF THE INVENTION

1. Field of the Invention

The present invention relates to a method for inhibiting nitric oxide (NO) and/or prostaglandin E₂, and in particular relates to a method for inhibiting NO and/or prostaglandin E₂ synthesis to relieve the syndromes of the inflammation.

2. Description of the Related Art

Nitric oxide (NO), prostaglandin E₂ (PGE₂), and associated enzymes have been identified as important mediators in the inflammatory processes. Large amounts of NO are produced by inducible nitric oxide synthases (iNOS), and PGE₂ is synthesized by cyclooxygenase (COX) (especially COX-2), respectively, during inflammation.

Nitric oxide (NO) is an unstable free radical that mediates both homeostatic and pathophysiologic processes within the cardiopulmonary, nervous, and immune systems and is synthesized by nitric oxide synthase. The list of potential disease associated with NO is increasing dramatically (Cochran et al., Medicinal Research Reviews, 1996, 16(6):547-563). Thus, agents that modulate the activity of NO may be of considerable therapeutic value. In particular, those that reduce the formation of NO may be beneficial in pathophysiological states in which excessive production of NO is a contributory factor. These include diseases such as septic shock, neurodegenerative disease, and inflammation.

Prostaglandins are a class of eiconisoids that play an important role in pain, fever and inflammation. They are synthesized from arachidonic acid, and possess a five-membered ring of carbon atoms that had formed part of the chain of arachidonic acid. Prostaglandins act locally, i.e., near the site of their synthesis. Prostaglandin E₂, is particularly relevant in causing fever, pain and inflammation (Funk, C. Science, 294:1871-1875 (2001)) and has been demonstrated to be an important pro-inflammatory and hyperalgesia-inducing lipid mediator. Reduction of excess prostaglandin E₂ may be beneficial in the diseases associated with inflammation such as arthritis, stroke, myocardial infarction, atheroma, urogenital disease, diabetes, renal function disorder and cancer.

Conventionally, it is expected that the compounds or agents capable of decreasing the amount of NO or PGE₂ or the activity of their associated enzymes iNOS and COX-2 are useful as therapeutic agents. For example, U.S. Pat. No. 5,266,594 discloses that inhibitors of nitric acid synthase can be used to prevent neurotoxicity mediated through glutamate receptors. Nitric oxide synthase inhibitors can be used therapeutically in the treatment of vascular stroke and neurodegenerative disorders such as Alzheimer's disease and Huntington's disease. U.S. Pat. No. 6,235,747 relates to certain 6-phenyl-pyridin-2-ylamine derivatives that exhibit activity as nitric oxide synthase (NOS) inhibitors, to pharmaceutical compositions containing them and to their use in the treatment and prevention of central nervous system disorders. U.S. Pat. No. 6,432,947 discloses pharmaceutical compositions containing compounds, methods of using these compounds as inhibitors of nitric oxide synthase and processes for synthesizing these compounds are also described herein, wherein the compound contains N-Heterocyclic derivatives. U.S. Patent No. 2005234030 discloses that certain substituted indoles that are modulators of COX-2 are useful for treatment of pain and/or inflammation as well as other disorders. U.S. Pat. No. 6552075B₂ discloses that certain substituted aryl compounds as COX-2 selective inhibitors are useful for chemoprevention.

However, no prior art suggests a new use of butylidene phthalide, citronellol, or geraniol for inhibiting the synthesis of nitric oxide and/or prostaglandin E₂.

### BRIEF SUMMARY OF INVENTION

The invention provides a method for inhibiting nitric oxide (NO) and/or prostaglandin E₂ synthesis, comprising administering a composition to a subject, wherein the composition comprises an effective amount of butylidene phthalide, citronellol, geraniol or a combination thereof, and a pharmaceutically acceptable carrier, salt or solvate.

The invention further provides a method to reduce or relieve the syndromes of the inflammation in a subject in which nitric oxide and/or prostaglandin E₂ production is implicated, comprising administering a composition to a subject, wherein the composition comprising an effective amount of butylidene phthalide, citronellol, geraniol or a combination thereof, and a pharmaceutically acceptable carrier, salt or solvate.

The invention further provides a method to reduce or relieve the disease of the inflammation in a subject in which nitric oxide and/or prostaglandin E₂ production is implicated, comprising administering a composition to a subject, wherein the composition comprising an effective amount of butylidene phthalide, citronellol, geraniol or a combination thereof, and a pharmaceutically acceptable carrier, salt or solvate.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIGS. 1a-1c show that butylidene phthalide, citronellol, or geraniol, respectively suppress the synthesis of nitric oxide;

FIGS. 2a-2c show that butylidene phthalide, citronellol, or geraniol, respectively suppress the synthesis of prostaglandin E₂;

FIGS. 3a-3c show that butylidene phthalide, citronellol, or geraniol does not affect cell viability.

### DETAILED DESCRIPTION OF INVENTION

The following description is of the best-contemplated mode of carrying out the invention. This description is made for the purpose of illustrating the general principles of the invention and should not be taken in a limiting sense. The scope of the invention is best determined by reference to the appended claims.

Essential oils, also called volatile or ethereal oils, are aromatic oily liquids obtained by expression, fermentation, enfleurage or extraction from plant material such as flowers, buds, seeds, leaves, twigs, bark, herbs, wood, fruits and roots, as referred in “Essential oil: their antibacterial properties and potential applications in foods—a review” (Sara Burt, International Journal of Food Microbiology 2004; 94: 223-53). Essential oils with hydrophobic properties allow easy skin penetration and absorption into the body following external topical use.

Butylidene phthalide is a monoterpenoid with a molecular formula of C₁₂H₁₂O₂ and molecular weight of 188.2 Da. Butylidene phthalide can be extracted from Angelica sinensis, Rhizoma Chuanxiong and considered to exhibit anti-spasmodie, anti-anginal anti-atherosclerotic, acaricidal, insecticidal and anti-platelet activity.

Citronellol is a monoterpene with a molecular formula of C₁₀H₂₀O and molecular weight of 156.3 Da. Citronellol can be extracted from Rose Rugosa, Pelargonium graveolens, or Cymbopogon and considered to exhibit insecticidal, anti-fungus and anti-conflict activity.

Geraniol is a monoterpene with a molecular formula is C₁₀H₁₈O and molecular weight of 154.2 Da. Geraniol can be extracted from Rose Rugosa, Pelargonium graveolens, or Cymbopogon and considered to exhibit anti-fungus and antiviral activity.

The invention provides a method comprising administering a composition including an effective amount of butylidene phthalide, citronellol, geraniol or a combination thereof. Butylidene phthalide, citronellol, geraniol or a combination thereof can effectively suppress the nitric oxide (NO) and/or prostaglandin E₂ synthesis.

Butylidene phthalide can be obtained from, but are not limited to, Dong quai or Rhizoma Chuanxiong, and citronellol and geraniol can be obtained from, but are not limited to, citronella (e.g. Cymbopogon spp, or Cymbopogon citrates), rose (e.g. Rosa hybrida Hort), geranium (e.g. Pelargonium graveolens), Citrus hystrix, Eucalyptus citriodora Hook, Flos Magnoliae, Calendula officinalis, Verbena officinalis, Murraya paniculata, Spirodela punctata, Rhododendron spp, Geranium robertianum, Nepeta cataria, Kalanchoe blossfeldiana, Rhodiolafastigiata or Cymbopogon Martini.

The term “effective amount” of the invention refers to the amount of composition required in order to suppress the synthesis of nitric oxide (NO) and/or prostaglandin E₂.

The term “subject” is intended to include humans and also other non-human mammals, such as monkeys, cows, goats, sheep, dogs, cats, rabbits, rats, or mice.

The term “pharmaceutically acceptable carrier, salt or solvate” can include solvents, dispersion media, coatings, antibacterial and antifungal agents, isotonic and absorption delaying agents, and the like. The pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Suitable salts for the components to be employed according to the present subject matter are also those with inorganic cations, for example alkali metal salts, in particular sodium, potassium, or ammonium salts, alkaline earth metal salts such as, in particular, the magnesium or calcium salts, as well as salts with bi- or tetravalent cations, for example the zinc, aluminum, or zirconium salts. Also contemplated are salts with organic bases, such as dicyclohexylamine salts; methyl-D-glucamine; and salts with amino acids, such as arginine, lysine, histidine, glutamine and so forth. Also, the basic nitrogen-containing groups can be quaternized with such agents as: lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others. Salt-forming agents, for example, low molecular weight alkylamines such as methylamine, ethylamine, or triethylamine can also be employed. Water or oil-soluble or dispersible products are thereby obtained.

In the invention, the concentration of butylidene phthalide can be from about 10 μM to 1000 μM of the composition, preferably, from about 250 μM to 1000 μM, more preferably, from about 500 μM to 1000 μM. The concentration of citronellol can be from about 10 μM to 750 μM of the composition, preferably, from about 250 μM to 750 μM, more preferably, from about 500 μM to 750 μM. The concentration of geraniol can be from about 10 μM to 1000 μM of the composition, preferably, from about 500 μM to 1000 μM, more preferably, from about 750 μM to 1000 μM. Because butylidene phthalide, citronellol, and geraniol are easily absorbed through the skin, the composition of the invention can readily diffuse and work into the body following external topical use.

The composition of the invention can be administrated alone, or in combination with second compounds or drugs resulting in not only inhibiting NO and/or prostaglandin E₂ synthesis but also enhance other compounds or drugs to penetrate into the body. The second agents comprise other anti-inflammatory drugs, such as non-steroidal anti-inflammatory drug (e.g. flurbiprofen, naproxen, nabumetone, sulindac, etodolac, celecoxib, rofrcoxib, ibuprofen, diflunisal, indomethacin, piroxicam, diclofenac sodium, acetylsalicylic acid, or ketoprofen). In the invention, the composition can be butylidene phthalide, citronellol, and/or geraniol. In one embodiment, the composition can be geraniol for inhibiting NO and prostaglandin E₂ synthesis. In another embodiment, the composition can be butylidene phthalide for inhibiting NO synthesis and further comprising geraniol for inhibiting prostaglandin E₂ synthesis.

The amount of active ingredient that can be combined with the carrier materials to produce a single dosage form will vary depending upon the subject and the particular mode of administration. The dosage required will vary according to a number of factors known to those skilled in the art, including, but not limited to, the compound or compounds used, the species of subject, and the size of the subject.

The composition of the invention can be orally administrated, or externally topically used, wherein the latter is preferred.

For external application, the composition is formed into creams, ointments, gels, sprays, lotions, skin tonics, shampoos or mousses as referred to above. Skin sprays are generally composed of aerosolized copolymers, such as polyvinylpyrrolidone, vinyl acetate and the like, and may also function as a setting lotion. Skin gel preparations are similar to sprays in composition, but are in gel and alcohol free form, and can coat the skin. Skin mousse is foam released under pressure from an aerosolized can. A skin care composition may be formulated as a hydrophobic or hydrophilic cream, ointment, gel, emollient, spray, lotion, skin tonic, shampoo or mousse as referred to above, suitably with additional ingredients suitable for use in skin care compositions of types known in the art, and such further ingredients can include petrolatum, waxes, lanolin, silicone, liposomes, vegetable, mineral oils, plasticizers, fragrances, preservatives, a penetration enhancing agent, a pH adjusting agent or other suitable ingredients for topical skin compositions. Such ingredients can moisturize skin, stabilize the active compound, increase drug-skin contact and local concentration, control drug slow release, and/or aid in decreasing skin breakage, preventing skin atrophy, fibrosis and infection, and promoting skin wound healing.

In one embodiment, the composition of the invention can be a suitable form, e.g. tablet, emission oil, ointment, pellet, powder, or liquid, depending on requirements.

Additionally, butylidene phthalide, citronellol, and geraniol are DOH (Department of Health, Taiwan) approved food additives so that the composition of the invention is very safe and does not cause biological damage, and the Examples of the invention also confirm that butylidene phthalide, citronellol, and geraniol do not reduce cells viability.

The invention further provides a method for reducing or relieving the syndromes of the inflammation in a subject in which nitric oxide and/or prostaglandin E₂ production is implicated, comprising administering a composition to a subject, wherein the composition comprising an effective amount of butylidene phthalide, citronellol, geraniol or a combination thereof, and a pharmaceutically acceptable carrier, salt or solvate.

The syndromes caused by NO and/or prostaglandin E₂, e.g. septic shock, inflammation, pain, fever, homeostatic process disorder, tissue injury, neurodegenerative disease, or neuron toxicity, can be mitigated by administering the composition including butylidene phthalide, citronellol, geraniol or a combination thereof.

The invention further provides a method for reducing or relieving the disease of the inflammation in a subject in which nitric oxide and/or prostaglandin E₂ production is implicated, comprising administering a composition to a subject, wherein the composition comprising an effective amount of butylidene phthalide, citronellol, geraniol or a combination thereof, and a pharmaceutically acceptable carrier, salt or solvate. The relieving disease of the inflammation comprises platelet aggregation deficiency, homeostatic process disorder, tissue injury, migraine, inflammatory diseases, stroke, acute and chronic pain, hypovolemic shock, traumatic shock, reperfusion injury, Crohn's disease, ulcerative colitis, septic shock, multiple sclerosis, AIDS associated dementia, neurodegenerative diseases, neuron toxicity, Alzheimer's disease, chemical dependencies and addictions, emesis, epilepsy, anxiety, psychosis, head trauma, adult respiratory distress syndrome (ARDS), morphine induced tolerance and withdrawal symptoms, inflammatory bowel disease, osteoarthritis, rheumatoid arthritis, dilated cardiomyopathy, acute spinal cord injury, Huntington's disease, Parkinson's disease, glaucoma, macular degeneration, diabetic neuropathy, diabetic ulcer or cancer.

### EXAMPLE

### Example 1

### Suppression of Nitric Oxide Synthesis by Butylidene Phthalide, Citronellal, and Geraniol

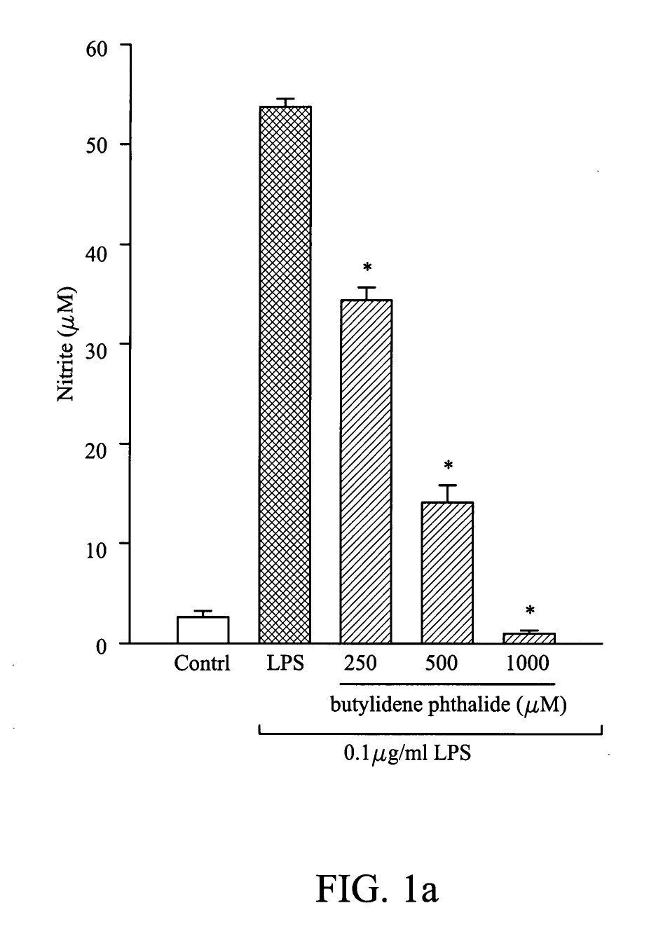

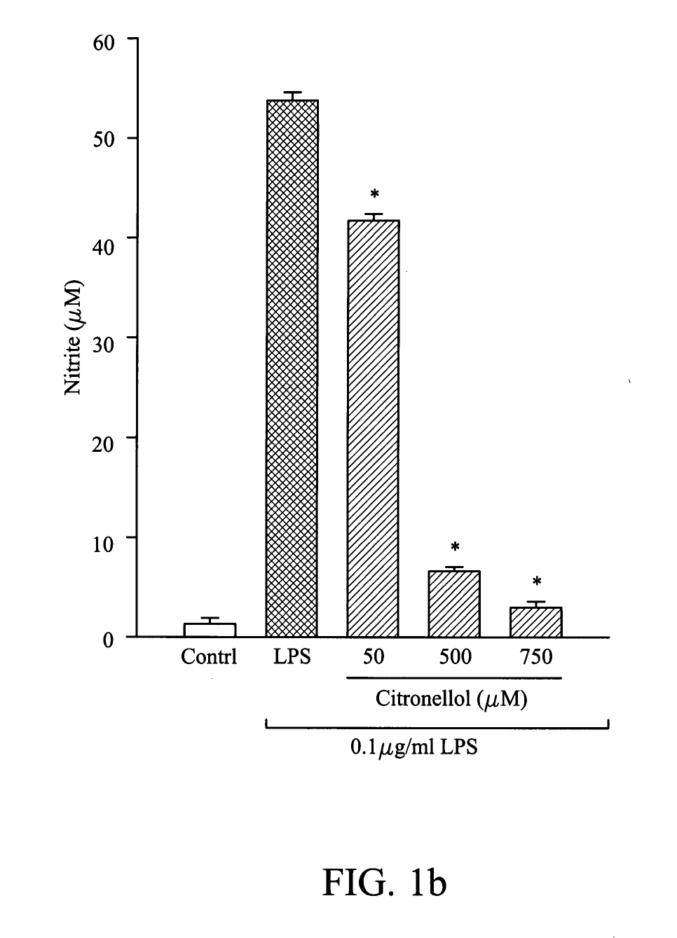

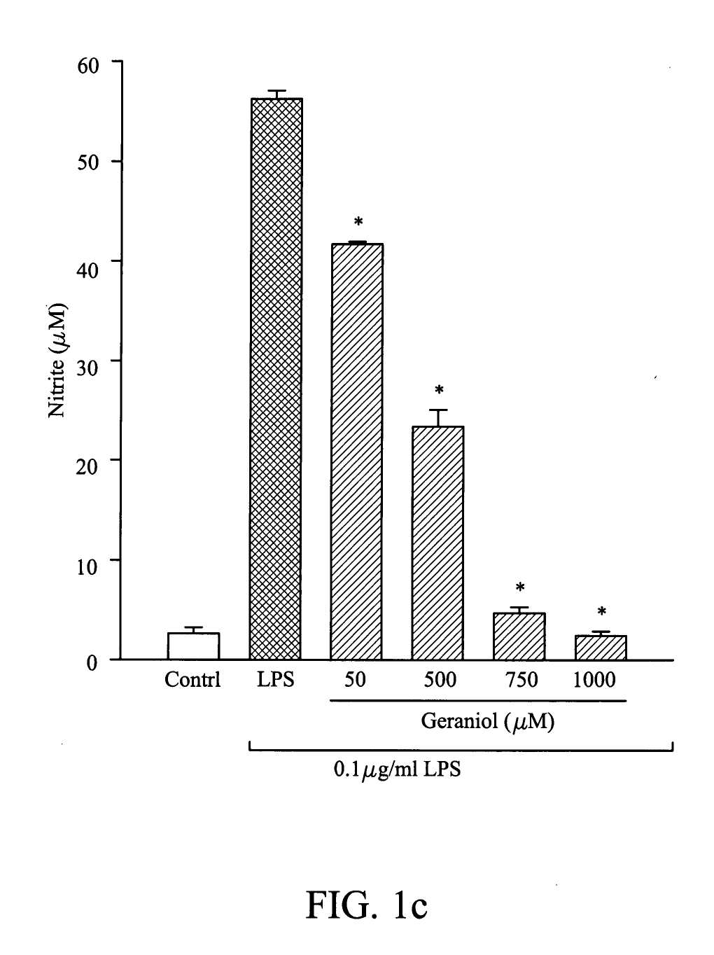

The murine macrophage/monocyte RAW 264.7 cells were obtained from the American Type Culture Collection (ATCC) and maintained in Dulbecco's modified Eagle's medium (DMEM) with 10% fetal bovine serum at 37° C. in 5% CO₂ humidified air. In this example, cells were plated at a density of 10⁶ cells/ml in 96-well plates for 24 hours, and then stimulated with LPS (100 ng/ml) in the presence of different concentrations of butylidene phthalide, citronellol, or geraniol. After 20 hours, the supernatant of the medium was collected and analyzed. In nitric oxide analysis, the concentration of butylidene phthalide was 250 μM, 500 μM, and 1000 μM, the concentration of citronellol was 50 μM, 500 μM, and 750 μM, and the concentration of geraniol was 50 μM, 500 μM, 750 μM, and 1000 μM, separately. In the control group, the cells were not treated with butylidene phthalide, citronellol, or geraniol. Because nitric oxide was unstable, nitrite was detected (Green et al., 1981; Kang et al., 1999a). 80 μl of the culture medium were mixed with an equal volume of Griess reagent (0.1% naphthylethylenediamine dihydrochloride and 1% sulfanilamide in 5% phosphoric acid), and then the absorbance at 550 nm was measured. The nitrite concentration was determined using a curve calibrated on sodium nitrite standards. According to the results of this experiment, the IC₅₀ (inhibitory concentration at 50%) of butylidene phthalide was 406.5 μM, the IC₅₀ of citronellol was 295 μM, and the IC₅₀ of geraniol was 402.8 μM. Referring to FIGS. 1a-1c, butylidene phthalide, citronellol, and geraniol can suppress the synthesis of nitric oxide, wherein the suppression ability of citronellol was better than butylidene phthalide or geraniol, separately.

### Example 2

### Suppression of Prostaglandin E₂ by Butylidene Phthalide, Citronellol, and Geraniol

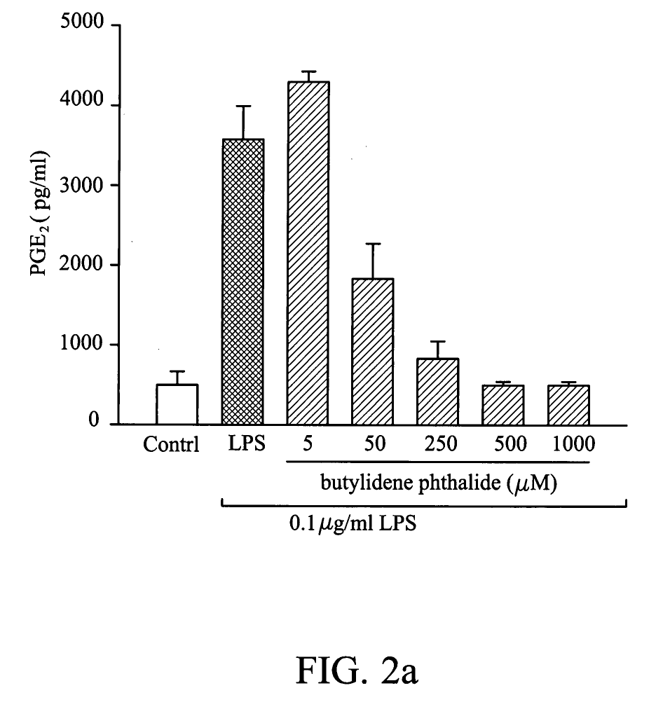

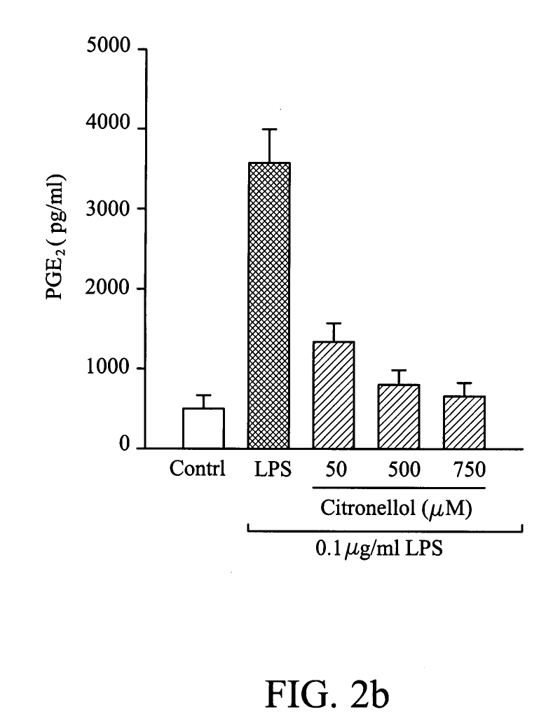

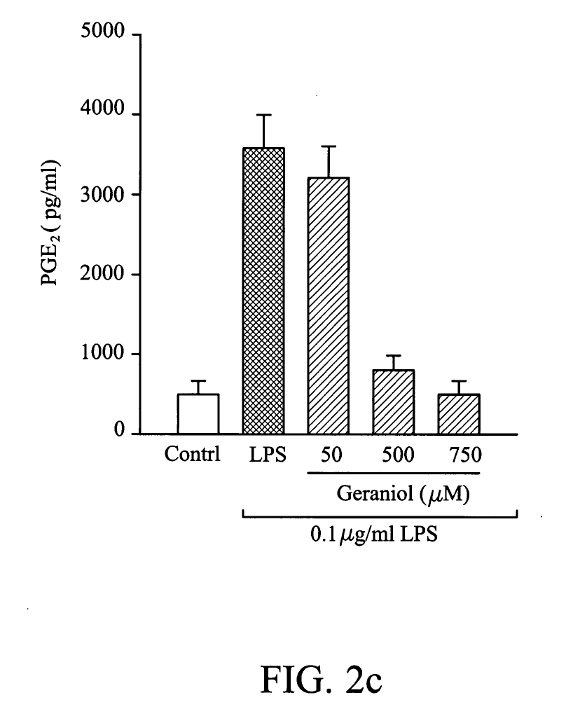

The same procedure carried out in Example 1 was repeated with the exception that the detection of the nitric oxide was changed to detect prostaglandin E₂ by prostaglandin E₂-monoclonal enzyme immunoassay kit (EIA, Cayman Chem., Ann Arbor, Mich.). The concentration of butylidene phthalide was 5 μM, 50 μM, 250 μM, 500 μM, and 1000 μM, the concentration of citronellol was 50 μM, 500 μM, and 750 μM and the concentration of geraniol was 50 μM, 500 μM, and 750 μM, separately. According to the results of this experiment, the IC₅₀ of butylidene phthalide was 49.6 μM, the IC₅₀ of citronellol was 26.88 μM, and the IC₅₀ of geraniol was 371.9 μM. Referring to FIGS. 2a-2c, synthesis of prostaglandin E₂ was suppressed in cells treated with butylidene phthalide, citronellol, and geraniol, wherein the suppression ability of citronellol was better than butylidene phthalide or geraniol, separately.

### Example 3

### Cell Viability Assay

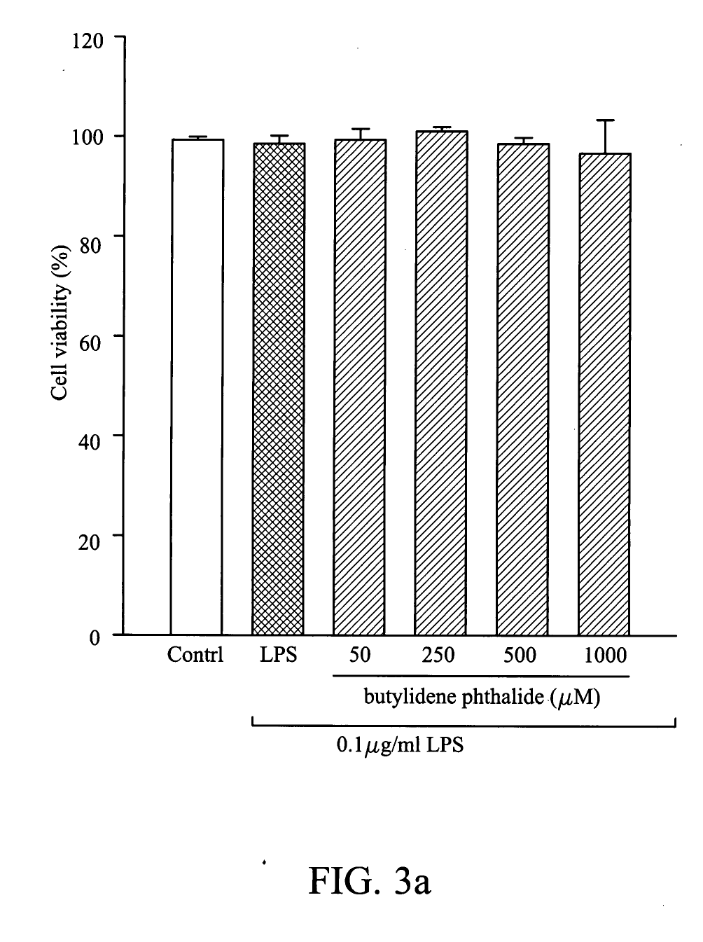

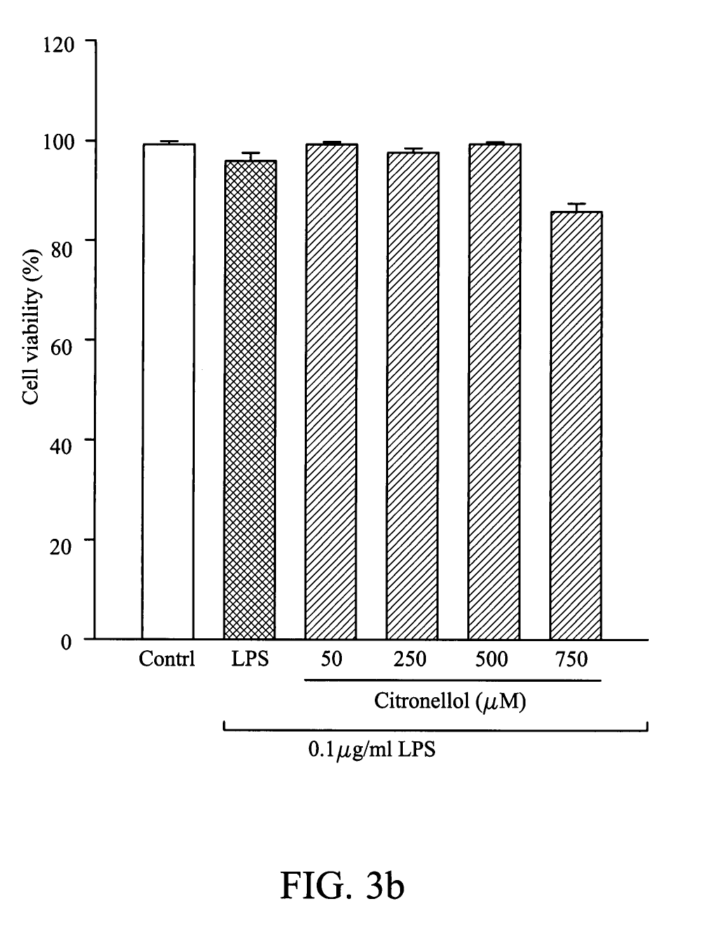

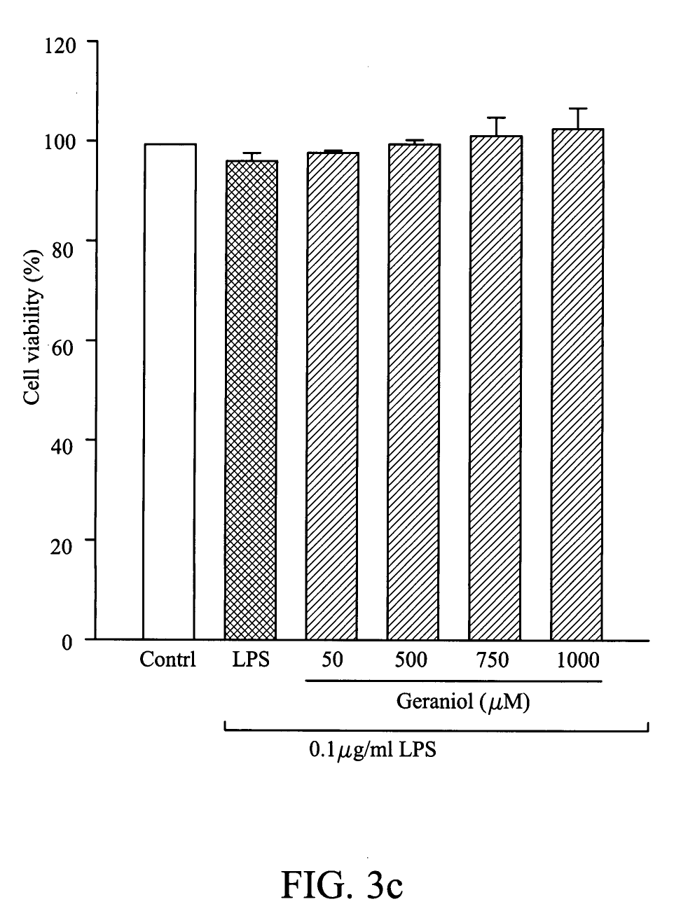

The same procedure carried out in Example 1 was repeated with the exception that the detection of the nitric oxide was changed to measure RAW264.7 cell viability by MTT assay. The concentration of butylidene phthalide was 50 μM, 250 μM, 500 μM, and 1000 μM, the concentration of citronellol was 50 μM, 250 μM, 500 μM, and 750 μM, and the concentration of geraniol was 50 μM, 500 μM, 750 μM, and 1000 μM, respectively. Referring to FIGS. 3a-3c, butylidene phthalide, citronellol, and geraniol did not affect cell viability.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for inhibiting and/or reducing nitric oxide (NO) and/or prostaglandin E₂ synthesis, comprising administering a composition to a subject, wherein the composition comprises an effective amount of butylidene phthalide, citronellol, geraniol or a combination thereof, and a pharmaceutically acceptable carrier, salt or solvate.

2. The method as claimed in claim 1, wherein the composition comprises a plant extract.

3. The method as claimed in claim 2, wherein the plant extract comprises Dong quai, Rhizoma Chuanxiong, citronella, rose or geranium extract.

4. The method as claimed in claim 1, wherein the composition comprises an effective amount of geraniol for inhibiting and/or reducing NO and prostaglandin E₂ synthesis.

5. The method as claimed in claim 1, wherein the composition comprises an effective amount of geraniol for inhibiting and/or reducing NO synthesis.

6. The method as claimed in claim 5, further comprising a second effective amount of butylidene phthalide for inhibiting and/or reducing prostaglandin E₂ synthesis.

7. The method as claimed in claim 1, wherein the composition comprises an effective amount of butylidene phthalide for inhibiting and/or reducing NO synthesis.

8. The method as claimed in claim 7, further comprising a second effective amount of geraniol for inhibiting and/or reducing prostaglandin E₂ synthesis.

9. The method as claimed in claim 1, wherein the concentration of butylidene phthalide is from about 10 μM to 1000 μM of the composition.

10. The method as claimed in claim 1, wherein the concentration of butylidene phthalide is from about 500 μM to 1000 μM of the composition.

11. The method as claimed in claim 1, wherein the concentration of citronellol is from about 10 μM to 750 μM of the composition.

12. The method as claimed in claim 1, wherein the concentration of citronellol is from about 500 μM to 750 μM of the composition.

13. The method as claimed in claim 1, wherein the concentration of geraniol is from about 10 μM to 1000 μM of the composition.

14. The method as claimed in claim 1, wherein the concentration of geraniol is from about 750 μM to 1000 μM of the composition.

15. The method as claimed in claim 1, wherein the composition is administered in oral or externally.

16. A method for reducing or relieving the syndromes of the inflammation in a subject in which nitric oxide and/or prostaglandin E₂ production is implicated, comprising administering a composition to a subject, wherein the composition comprising an effective amount of butylidene phthalide, citronellol, geraniol or a combination thereof, and a pharmaceutically acceptable carrier, salt or solvate.

17. The method as claimed in claim 16, wherein the syndromes of the inflammation comprises fever, pain, edema, homeostatic process disorder, tissue injury or shock.

18. A method for reducing or relieving the disease of the inflammation in a subject in which nitric oxide and/or prostaglandin E₂ production is implicated, comprising administering a composition to a subject, wherein the composition comprising an effective amount of butylidene phthalide, citronellol, geraniol or a combination thereof, and a pharmaceutically acceptable carrier, salt or solvate.

19. The method as claimed in claim 18, wherein the disease of the inflammation comprises platelet aggregation deficiency, homeostatic process disorder, tissue injury, migraine, inflammatory diseases, stroke, acute and chronic pain, hypovolemic shock, traumatic shock, reperfusion injury, Crohn's disease, ulcerative colitis, septic shock, multiple sclerosis, AIDS associated dementia, neurodegenerative diseases, neuron toxicity, Alzheimer's disease, chemical dependencies and addictions, emesis, epilepsy, anxiety, psychosis, head trauma, adult respiratory distress syndrome (ARDS), morphine induced tolerance and withdrawal symptoms, inflammatory bowel disease, osteoarthritis, rheumatoid arthritis, dilated cardiomyopathy, acute spinal cord injury, Huntington's disease, Parkinson's disease, glaucoma, macular degeneration, diabetic neuropathy, diabetic ulcer or cancer.
